(19) United States
(12) Patent Application Publication
(10) Pub. No.: US 2006/0094067 A1
(43) Pub. Date: May 4, 2006

(54) Method for the determination of characteristics and/or the classification of circulating macrophages, and analysis arrangement for carrying out said method

(76) Inventor: Ralf Herwig, Westendorf (AT)

Correspondence Address: MERCHANT & GOULD PC, P.O. BOX 2903, MINNEAPOLIS, MN, 55402-0903, US

(21) Appl. No.: 10/519,174

(22) PCT Filed: Jun. 2, 2003
(86) PCT No.: PCT/EP03/05763

§ 371 Date: Sep. 1, 2005

(30) Foreign Application Priority Data
Jun. 26, 2002 (DE) 102 28 548.9
Jul. 9, 2002 (DE) 102 30 893.4

(51) Int. Cl. G01N 33/574; C12M 1/34

(52) U.S. Cl. 435/7.23; 435/287.2

(57) ABSTRACT

The invention relates to a method and an analysis arrangement for the determination of characteristics and/or classification of circulating macrophages. A whole blood sample is subjected to a gradient centrifugation for isolating macrophages. The macrophage cells are then perforated and provided with an intracellular staining with at least one selected antibody. A flow cytometric analysis of the pre-treated cells enables a subsequent statistical evaluation of the cell contents. PSA, a cytokeratin and/or an epithelial membrane antigen are selected as antibodies.

The invention relates to a method for determination and/or classification of circulating macrophages by heterologous antigens and to an analysis arrangement for carrying out such a method.

From Sinha, Wilson, Gleason: Immunoelectron microscopic localization of prostatic-specific antigen in human prostate by the protein A-gold complex, Cancer, 1987, 60, 1288-91, it is known to carry out electron microscope analyses of cells from prostate tissues, wherein according to the cited reference normal prostate tissue, prostate carcinoma tissue and prostate hyperplasia tissue were incubated with gold labeled PSA antibodies. The analyses revealed that the gold particles are located in the cytoplasm, in intracellular granules, the RES and lysosomes. With increasing terminal differentiation of the tumor, more gold particles appear in membrane structures. This was taken as an indication that with increasing terminal differentiation of the tumor cells, PSA (prostate-specific antigen) is incorporated in membrane structures. In a further aspect of this analysis gold particle were also recognized in granulocytes and macrophages.

By flow cytometric analyses, PSA positive cells were found in circulating blood. However, in the prior art analyses only the surfaces of macrophages were stained for PSA.

The fact that no mRNA of the PSA molecule was found in macrophages, leads to the sole conclusion that only the PSA molecule is taken up and that there is no elimination of micro metastases. It is referred to Brandt, Griwatz, Brinkmann: Circulating prostate-specific antigen/CD14-double-positive cells; a biomarker indicating low risk for hematogeneous metastasis of prostate cancer, J. Natl. Cancer Inst. 1997; 89, 174.

It is known that malignant changes of tissue-connected cells, which are gathered in a more or less ordered cell duster, are referred to as tumor. These tumor cells disregard the tissue order, they grow unrestricted and they expand by an increase of size and by infiltration into the surrounding tissue, organ, or they grow beyond the organ boundaries into the blood stream and the lymphoid system.

Once the tumor has reached the blood stream or the lymphoid system, single cells or cell clusters may be floated away by these systems, and the cells can adhere as metastases, i.e. metastases at different sites of the body. There is existing danger that the metastases grow further and consume energy of the body until the body deteriorates and is consumed by its disease.

During the development of such a tumor, the tumor cells will produce substances, which serve to assist in this growth. Additionally, substances may be released, which can be used as a marker for tumor growth. The latter are called tumor markers. However, these markers are not specific for a tumor, but only the amount of the measured concentration in blood, because healthy cells may also release such substances. Therefore, tumor markers cannot be used for the detection of a tumor, but only for control of the progress of the disease or therapy. A specific marker for a tumor is the prostate-specific antigen (PSA), which indicates a prostate carcinoma when found in a certain concentration in blood. However, a benign growth of the prostate may also give rise to an increase of the PSA value in blood.

Up to now, tumor diseases are diagnosed mainly by picture-based methods, like ultrasound or computer tomography, mammogram, etc. However, a definite decision is made only after a tumor-positive tissue sample and the determination of the therapy schedule.

The immune system of the human body is directed against tumor diseases. This immune system consists of a series of different cell types, which fulfill different functions. Among others, macrophages need to fulfill the task to recognize and phagocyte abnormal material, and to disintegrate the material in its components. Subsequently, fragments of cells taken up are presented on the surface of other immune cells, to give them the possibility to recognize the structure, against which the reaction shall be directed.

There is a strong need to conduct a determination of characteristics of circulating macrophages at an early stage, without the need to carry out examinations directly on the human body.

It is the object of the invention to provide a method and an analysis arrangement which allows a determination of characteristics and/or classification of circulating macrophages (PBMC).

According to the invention, it is believed that antigens or fragments of phagocyted tumor cells can be detected in circulating macrophages so that a direct and specific tumor detection is possible.

According to the invention, a whole blood sample is taken and a subsequent gradient centrifugation for the isolation of macrophages is carried out. The macrophage cells are then perforated, and the cells are intracellularly stained with at least one selected antibody.

Subsequently, per se known flow cytometry is used in order to record the cell characteristics on a single level.

Flow cytometry allows counting and analysis of physical and molecular characteristics of cells in a liquid flow. Precisely, with the help of samples marked with a fluorescent dye, e.g. antibodies, a determination of the characteristics of cells or populations of cells is carried out on a single level, and is recorded.

The antigen antibody reaction, which is carried out with the help of antibodies marked with a fluorescent dye, serves as a basis. For analysis, the cells of a single suspension are guided along a coherent laser beam with an appropriate wavelength by hydrodynamic focusing. After excitation of electrons of the fluorescent dye by the monochromatic laser beam, the electrons are shifted to an elevated energy level. After the laser pulse, the electrons return to their base level while emitting energy in form of photons. The emitted photon concentration, which is detected by a photo detector, is proportional to the amount of antibodies bound to each cell. Additionally, information on the cell size and the internal structure, i.e. the granular structure of the cytoplasm, the size of the nucleus etc., are gained by deflected and scattered light.

As selected antigens prostate-specific antigens, cytokeratin antibodies and/or epithelial membrane antigen are used.

According to the invention, by staining of the PSA antibody in the macrophages, it can be determined, whether the phagocyted material is prostate relevant.

The analysis arrangement for carrying out the method comprises means for heparinizing drained blood, a gradient centrifuge for isolating macrophages, means for cell perforation, a device for intracellular staining of said pre-treated cells with fluorochrome antibodies and a flow cytometer comprising a computer supported evaluation unit for determining the intracellular structure of the isolated and pretreated cell for the purpose of early diagnostic of tumors.

The invention will be further illustrated in the following by means of an embodiment.

In the step of taking blood and staining, for example 6 ml whole blood are used, which is subjected to heparinization. With the help of gradient centrifugation monocytes, macrophages and lymphocytes are isolated.

In the next step, a formaldehyde fixation and treatment of the cells with saponine is carried out for perforation.

Subsequently, the step of intracellular staining with selected antibodies, e.g. of the following table, is carried out.

PSA-antibody Ab-1 (Clone ER-PRS)

Pan-cytokeratin-FITC

Epithelial membrane antigene (Clone E 29)

Isotype control IgG1 (Clone DAK-GO1)

Secondary antibody FITC goat anti mouse (DAKO)

Until analysis the cell is again fixed and is then characterized by flow cytometry. Monocytes and macrophages are gated, i.e. only a portion of the measurement results is used for evaluation, and a pre-choice is made.

Subsequently, the isotype control and the staining are evaluated by histogram analysis, and the amount of positive cells, e.g. as percentage, is given.

It has been shown that in patients with scattered prostate tumor, parts of the structure of tissue cells can be found in the circulating immune cells of the respective person; if macrophages are stained with cytokeratin. As these elements are no original contents of the immune cells, they must have been taken up by phagocytosis. An unspecific effect can be excluded as the recorded curve progression of cytokeratin is clearly distinct from the curve progression of the isotope.

The staining of PSA in macrophages proves that the phagocyted material is prostate tissue, as this specific marker is also detectable.

In summary, the described method and the accompanying analysis arrangement provide a novel method for determination of characteristics and classification of circulating macrophages, wherein the classification allows indications on possibly prostate relevant facts.

1. Method for determination of characteristics and/or classification of circulating macrophages and/or peripheral mononuclear blood cells comprising the steps of:
taking whole blood and gradient centrifugation for isolating macrophages, peroration and said macrophage cells,
intracellular staining of said cells with at least one selected antibody and
flow cytometric analysis of said pre-treated cells comprising subsequent statistical evaluation of a plurality of cells.

2. Method of claim 1, wherein the use of prostate-specific antigen (PSA), cytokeratin and/or epithelial membrane antigen as said selected antibody/ies.

3. Method of claim 1 wherein histogram analysis of the isotype control and staining after carrying out flow cytometry.

4. Method of claim 1 for detecting parts of tissue cells uptaken by phagocytosis of a scattered prostate tumor outside the human body.

5. Method of claim 4, wherein it is determined by said staining of PSA in said macrophages, whether said material taken up by phagocytosis is prostate relevant.

6. Analysis arrangement of carrying out said method of, claim 1 comprising means for heparinizing drained blood, a gradient centrifuge for isolating macrophages, means for cell perforation, a device for intracellular staining of said pretreated cells with fluorochrome antibodies and a flow cytometer comprising a computer supported evaluation unit for determining the intracellular structure of the isolated and pretreated cell for the purpose of early diagnostic of tumors.
